(19) United States
(12) Patent Application Publication
(10) Pub. No.: US 2016/0089274 A1
(43) Pub. Date: Mar. 31, 2016

(54) LAPAROTOMY SPONGES DETECTABLE IN A MAGNETIC FIELD

(71) Applicants: John Hunter, Portland, OR (US); Erin W. Gilbert, Portland, OR (US); Joshua K. Hoyt, Portland, OR (US); Peter Galen, Portland, OR (US)

(72) Inventors: John Hunter, Portland, OR (US); Erin W. Gilbert, Portland, OR (US); Joshua K. Hoyt, Portland, OR (US); Peter Galen, Portland, OR (US)

(21) Appl. No.: 14/863,975

(22) Filed: Sep. 24, 2015

Related U.S. Application Data
(60) Provisional application No. 62/055,501, filed on Sep. 25, 2014.

(51) Int. Cl. A61F 13/44; A61F 13/36

(52) U.S. Cl. CPC A61F 13/44; A61F 13/36

(57) ABSTRACT

Laparotomy sponges comprising one or more closed metal wire loops incorporated into the fabric of the sponge are disclosed. The wire is of a gauge that is detectable by a metal detector. At least 50% of the area of the sponge is circumscribed by the one or more closed metal wire loops.

### FIELD

Generally, the field of the invention is surgical instruments. More specifically, the field is laparotomy sponges that are readily detectable after surgery.

### BACKGROUND

The retention of an object in a patient following surgery occurs at least once in every 5000-7000 surgeries. Such errors result in significant patient complications and liability costs. Laparotomy sponges are some of the most commonly retained surgical objects. Commonly, sponges are tracked through the use of manual counting before and after surgery. A failure of the post-surgery count to match the initial count results in surgical staff expending valuable time and effort locating the sponge.

In some examples, a detectable material such as a radiopaque material or a metal wire is incorporated into the sponge. This allows surgical staff to locate the sponge using a metal detector, x-ray machine, or other appropriate device. While an X-ray is capable of pinpointing the location of a retained surgical object, particularly a surgical object made of or labeled with metal or another radiopaque material, the machines are not easily or efficiently housed in an operating room therefore require time in transporting the X-ray machine to the patient. Metal detectors may be sufficiently small to house in an operating room, but currently available labels in sponges are not efficiently detected using a metal detector.

The Safety Sponge™ System, developed by SurgiCount, includes individually and uniquely bar-coded surgical sponges. Hand held sight scanners are used to scan and count sponges “in” at the beginning of surgery and “out” at the end of surgery. There are several limitations of this system. First, this system was developed only for the use of sponges. Manual counts are still relied on for all other surgical instruments, which account for ⅔ all of retained foreign objects. Additionally, this system cannot identify where the left behind sponges are located.

The SmartSponge™ System, developed by ClearCount, consists of a SmartBucket™ with SmartWand™ and SmartSponges™. The SmartBucket™ records and stores your counts of Radiofrequency Identification (RFID) tagged SmartSponges™ counts with computer assistance to establish your initial baseline and final counts. The SmartWand™ can be used to perform a scan of the patient to identify where the left behind sponge is located. There are several limitations of this system, as well. This system was only developed to identify retained RFID tagged sponges. There is the potential of markers falling off of these sponges. Additionally, sponges only account for ⅓ of all retained foreign objects. Manual counts are still relied on for all other surgical instruments. RF also can severely interfere with and shut down critical care operating room devices.

The RF Surgical™ System consists of a handheld scanning wand connected to a compact, self-calibrating console. Micro passive RF tags are embedded in a variety of surgical gauze, sponges and towels. Used in conjunction with manual counting procedures, the system will be activated with an audible and visual alert, when the wand is passed over a patient and any retained object fitted with a tag is present. The limitations of this system are that it was developed to identify retained sponges, which account for ⅓ of all retained foreign objects. There is the potential of tags falling off of these sponges. Manual counts are still relied on for all other surgical instruments. RF interference also severely interferes with and shut down critical care operating room machines.

Clearly an efficient system for the detection of retained surgical objects that includes both sponges and metal surgical instruments is necessary.

### SUMMARY

Disclosed herein is a laparotomy sponge that uses embedded wire loops incorporated into each sponge to dramatically enhance their detectability using conventional metal detection sensors and circuits. One or more closed loops of wire embedded in the sponge provide the following benefits:
The sponge retains its flexibility and absorption characteristics.
The use scenario by surgeons and nurses in the OR remain unchanged from current practice.
Multiple loops allow the sponge to be wadded up without losing detection sensitivity.
The wire is radio-opaque, allowing for optional X-ray detection.
Multiple loops provide redundancy in the event that a loop is “broken.”
The same detection circuit can be used to find surgical instruments left in the body cavity.

Use of the disclosed laparotomy sponge in surgery allows a simple “sweep” of a metal detector over the patient following surgery to detect the presence of the sponges within the patient, particularly when a manual count results in the possibility of a sponge retained in the patient.

### BRIEF DESCRIPTION OF THE DRAWINGS

FIG. 1 is a top view of one example of the disclosed laparotomy sponge comprising a rounded wire loop.

FIG. 2 is a top view of another example of the disclosed laparotomy sponge comprising two rounded wire loops in the same plane.

FIG. 3 is a top view of another example of the disclosed laparotomy sponge comprising a rectangular wire loop.

FIG. 4 is a top view of another example of the disclosed laparotomy sponge comprising two rectangular wire loops in the same plane.

FIG. 5 is a top section of another example of the disclosed laparotomy sponge comprising two loops arranged in a stack.

### DETAILED DESCRIPTION

Disclosed is a laparotomy sponge comprised of multiple metal wire loops incorporated into the fabric of the sponge. The wire making up the loop is of a gauge and type that is efficiently detected by a metal detector. At least 50% of the total area of the pad is encompassed by the one or more closed metal wire loops.

A laparotomy sponge or laparotomy pad is an absorbent pad used in surgical procedures and is commonly included in surgical kits laid out in an operating room in preparation for surgery. Laparotomy sponges are manufactured in any of a number of sizes including 4″×18″, 12″×12″, 18″×18″, 18″×36″. Laparotomy sponges can include fabric loops or ties in one or more of the corners to aid in handling the sponge. They may be provided in sterilized containers or packs and may be incorporated with radio-opaque tapes or strands of thread that allow detection of the sponges using an x-ray machine. A laparotomy sponge may be made of any appropriate absorbent material including cotton or synthetic materials.

A closed metal wire loop provides increased sensitivity in the alternating magnetic field of a metal detector relative to a wire that does not form a loop. The closed metal wire loop forms at least one complete loop such that the wire is curved such that at least a first point on the wire is in joined to at least a second point on the wire, thereby forming a closed loop. The loop can be formed by joining one end of the wire to the other end of the wire such that the loop has no wires extending outside of the loop. Alternatively, the loop can be formed by joining a first point that is not at the end of the wire to a second point that may be any other point on the wire (including an end.) In such a configuration, one or more lengths of wire extend outside of the loop from the point at which the first and second points are in contact.

The wire may be of any metal or alloy thereof, preferably a conductive, flexible metal that will not snap or break when folded or deformed such as in the situation where the sponge is wadded up. The wire may include iron, cobalt, copper, aluminum, tungsten, zinc, nickel, platinum, titanium, silver, gold, or any alloy that is a combination of any of these, or any other appropriate flexible material. The wire may be solid, stranded, or braided. The wire may be coated with an insulating or other material (such as plastic), including a radiopaque material that can be readily visualized in an X-ray image.

In some examples, the wire out of which the loop is formed is of a gauge that is efficiently detected by a metal detector. For example, wires that can be used include 16 gauge or smaller, 18 gauge or smaller, 20 gauge or smaller, 22 gauge or smaller, 24 gauge or smaller, 26 gauge or smaller, 28 gauge or smaller, or in ranges between 16 and 28 gauge, between 18 and 26 gauge, between 20 and 24 gauge, between 21 and 23 gauge, or 22 gauge (values given in American wire gauge).

In further examples, the one or more wire loops circumscribe at least 50%, at least 60%, at least 70%, at least 80%, at least 90%, at least 95%, at least 99% or more of the surface area of the sponge. In some examples a single loop circumscribes the area. In other examples the total of two or more loops circumscribes the area. For example, multiple loops, each of which circumscribes less than 50% of the area of the sponge may, in total circumscribe more than 50% of the area of the sponge.

The one or more wire loops can be configured to form any 2-dimensional shape including a circular shape, an oval shape, a triangular shape, a star shape, or a shape comprising one or more right angles such as a rectangle or square. A sponge may comprise wire loops of two different shapes. For example, the sponge may comprise a circular wire and a square wire.

The wire can be incorporated with the sponge by any method. It can be woven into the sponge, adhered to the sponge via an adhesive, or stitched to the sponge through any of a number of methods. In one such method, the wire is sandwiched between a patch made of a flat, flexible material such as a fabric and the sponge and stitching the fabric to the sponge thereby encasing the wire within the patch. Alternatively, the sponge can be made with one or more pockets into which wires can be inserted. The pockets can be stitched closed. In another alternative, the wire can be placed upon the surface of the sponge and a set of stitches used to incorporate the wire with the sponge. These stitches can form an X-pattern, an H-pattern or any other pattern that attaches the wire to the sponge.

### EXAMPLES

Provided herein are nonlimiting examples of various embodiments of the invention.

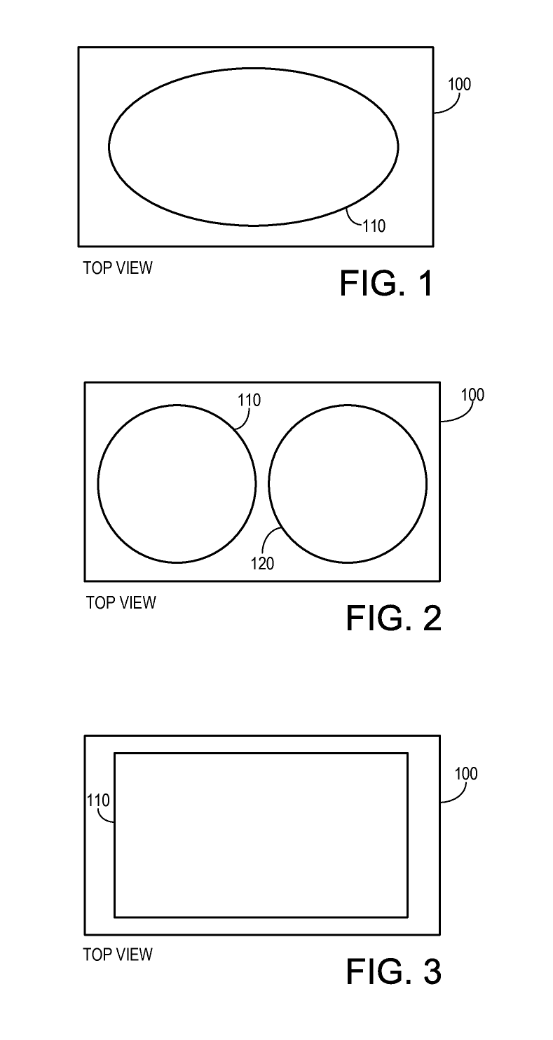

FIG. 1 is a top view of a sponge 100 comprising a wire loop 110. In this Figure, the wire loop is in the shape of an oval.

FIG. 2 is a top view of a sponge 100, comprising a first wire loop, 110 and a second wire loop 120. In this Figure, both the first wire loop and the second wire loop are circles. While neither individual wire loop encompasses at least 50% of the area of the sponge, the combination of the two circles does encompass at least 50% of the area of the sponge.

FIG. 3 is a top view of a sponge 100 comprising a wire loop 110. In this Figure, the wire loop is in the shape of a rectangle.

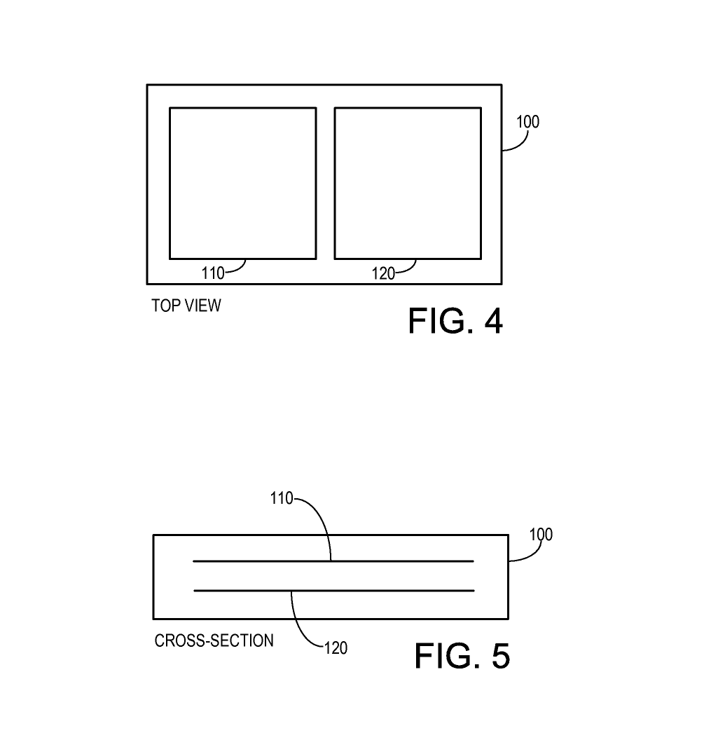

FIG. 4 is a top view of a sponge 100 comprising a first wire loop 110 and a second wire loop 120. In this Figure, both the first wire loop and the second wire loop are squares.

FIG. 5 is a cross section of a sponge 100, comprising a first wire loop 110 and a second wire loop 120. The wire loops are arranged in a stack relative to one another.

1. A laparotomy sponge comprising:
one or more closed metal wire loops incorporated into the fabric of the sponge, said metal wire loop being of a gauge detected by a metal detector, wherein at least 50% of the area of the sponge is circumscribed by the one or more closed metal wire loops.

2. The laparotomy sponge of claim 1 comprising two or more closed metal wire loops.

3. The laparotomy sponge of claim 2 wherein the two or more closed metal wire loops are arranged in the same plane relative to one another.

4. The laparotomy sponge of claim 2 wherein at least two of the closed metal wire loops are arranged in a stack relative to one another.

5. The laparotomy sponge of claim 1 wherein at least one of the closed metal wire loops forms a circle.

6. The laparotomy sponge of claim 1 wherein at least one of the closed metal wire loops forms a rectangle.

7. The laparotomy sponge of claim 1 wherein the gauge of the wire is between 18 and 30.

8. The laparotomy sponge of claim 7 wherein the gauge is between 21 and 23.

9. The laparotomy sponge of claim 1 wherein the one or more closed metal wire loops circumscribes at least 90% of the area of the sponge.
